(19) United States
(12) Patent Application Publication
(10) Pub. No.: US 2008/0124275 A1
(43) Pub. Date: May 29, 2008

(54) Crystalline Form of Fatty Acid Amide Hydrolase (Faah)

(75) Inventors: Michael H. Bracey, San Diego, CA (US); Michael A. Hanson, Del Mar, CA (US); Raymond C. Stevens, La Jolla, CA (US); Benjamin F. Cravatt, La Jolla, CA (US)

Correspondence Address: Olson & Cepuritis, LTD., 20 NORTH WACKER DRIVE, 36TH FLOOR, CHICAGO, IL, 60606, US

(73) Assignee: The Scripps Research Institute

(21) Appl. No.: 10/534,766

(22) PCT Filed: Nov. 14, 2003
(86) PCT No.: PCT/US03/36125

§ 371 Date: Jun. 2, 2006

Related U.S. Application Data
(63) Continuation-in-part of application No. 60/426,788, filed on Nov. 14, 2002.

(51) Int. Cl. A61K 47/00; C12N 9/14; G06G 7/48; A61P 37/00; A61P 43/00; A61P 1/00; A61K 49/00; C12Q 1/34

(52) U.S. Cl. 424/9.1; 435/195; 703/11; 435/18; 514/789

(57) ABSTRACT

The present invention is directed to FAAH crystals in complex with the inhibitor methoxyarachidonyl fluorophosphonate (MAFP) and to the use of these crystals to determine the three-dimensional structure of FAAH. This invention id further directed to the use of this structure for the modeling or determination of the structures of related proteins. This invention is further directed to the use of this structure in the pursuit of drug design to identify, characterize, or optimize agents which bind to the active site, substrate channels, product channels, or regulatory sites of FAAH, and to the evaluation of these agents to identify agents which may stimulate, inhibit, relocalize, stabilize, or destabilize FAAH and/or its activity. This invention is further directed to the use of this structure in the development of engineered FAAH variants which display altered solubility, catalytic profiles, or substrate specificity. This invention is further directed to the use of this structure in the development of engineered heterologous proteins with altered membrane tropism.

### GOVERNMENT SUPPORT

This application was partially supported by National Institutes of Health Grant Nos. DA13173 and F32 MH12414-03. Accordingly, the U.S. Government may have an interest in this application.

### FIELD OF THE INVENTION

The present invention relates generally to structural biology and medicine, especially in the interception of endocannabinoid influence and allied physiological processes. More specifically, the present invention relates to the crystalline form of fatty acid amide hydrolase (FAAH) and the use of these crystals to determine the three-dimensional structure of this protein.

### BACKGROUND OF THE INVENTION

Fatty acid amide hydrolase (FAAH) is the only characterized mammalian member of the amidase signature (AS) family of serine hydrolases. This family is an ancient and ubiquitous group of enzymes that share an amino acid motif known as the amidase signature. The representatives of this family have a highly diverse array of substrates though they may share a common reaction mechanism. Most AS enzymes described to date hydrolyze amides of small metabolic intermediates, such as acetamide, opines, propionamide, and malonamide. Those AS enzymes described to date also appear to be exclusively soluble proteins.

Though a member of the AS family, FAAH possesses unique features which distinguish it from lower homologues. FAAH behaves as an integral membrane protein when isolated from native sources or when expressed recombinantly. The enzyme cannot be separated from membrane fractions with the use of high salt concentrations or alkaline sodium carbonate. It can only be separated from membranes with the use of detergents. FAAH protein extracted with the aid of non-denaturing detergents retains catalytic activity and hence presumably its native structure.

FAAH is present in mammals in various tissues throughout the body, including the brain, liver, duodenum, kidney, and testis. The enzyme is noticeably absent from the heart. In these tissues, the enzyme appears to reside on extensive intracellular membrane systems, likely the smooth endoplasmic reticulum (SER). This conclusion is supported by confocal immunofluorescence data from each of these tissues as well as immunogold electron microscopy of the rat liver. The mechanism by which FAAH is inserted into these membranes and how it is anchored there is currently a subject of active investigation.

Unlike its metabolic counterparts in lower organisms, FAAH retains an important role in nervous system function. The substrates of FAAH, the fatty acid amides, have been demonstrated to exert powerful neuromodulatory effects in test animals relating to the physiologies of pain, locomotion, memory, cognition, pyresis, and sleep. The mechanism by which these compounds exert their influence is not yet fully characterized, though a subset of these effects can be abrogated in vivo by antagonists of the trimeric G-protein coupled receptor CB1. The fatty acid amides are hydrolyzed by FAAH in an expeditious manner to their pharmacologically inactive acids. Therefore, FAAH acts to terminate the signaling of these molecules and to establish their baseline levels in the cell. As a result, FAAH intersects the physiologies associated with its substrates.

In vivo demonstrations of the critical role that FAAH plays in mammals has recently been documented with the aid of a genetically engineered mouse model that lacks a functional FAAH gene. These mice possess greatly enhanced sensitivities to the neurological effects of the fatty acid amides when administered by intraperitoneal injection. Further, these mice exhibit an increased tolerance for pain even in the naïve state. Concomitantly, brains from these mice exhibit highly elevated levels of fatty acid amides, suggesting that an absence of FAAH leads to disturbed endogenous fatty acid amide function and results in altered baseline physiology. These observations suggest that a pharmacological intervention of the catalytic activity of FAAH may result in similar effects as the present genetic ablation.

The catalytic mechanism by which FAAH hydrolyzes its substrates has been investigated and several important findings have resulted. The amino acid residues that are responsible for the active site chemistry have been identified, as has at least one amino acid residue which resides in the substrate binding pocket. One particular finding of interest is the observation that FAAH is able to catalyze the hydrolysis of amides in the presence of equivalent amounts of comparable esters. This feature is contrary to solution chemistry and the action of the unrelated serine hydrolases of the catalytic triad family. How FAAH maintains this preference is currently unknown, though at least one amino acid residue, lysine 142, has been identified which contributes to this effect.

### SUMMARY OF THE INVENTION

The present invention provides a crystallized mammalian fatty acid amide hydrolase (FAAH) in complex with the active site-directed inhibitor methoxyarachidonyl fluorophosphonate (MAFP). The present invention further provides derivatives of invention FAAH crystals including various heavy metals and methods for the collection of the X-ray diffraction patterns of both native and derivative crystals. The invention also provides methods for analyzing diffraction patterns produced by invention native and derivative crystals by multiple isomorphous replacement (MIR) and single- and multiwavelength anomalous diffraction (SAD/MAD).

In one embodiment, there is provided a three-dimensional model for the protein structure of FAAH at the secondary, tertiary, and quaternary levels. Identification of this structural model allows the analysis of FAAH's physicochemical properties and its application to understanding the enzyme's function and mechanism in vivo.

In another embodiment, there are provided methods for the identification of the active site of FAAH as well as its substrate binding residues. The invention provides the unambiguous localization of the MAFP molecule within the FAAH protein structure, and therefore allows the assignment of amino acid residues that interact with the enzyme's natural substrates as mimicked by the arachidonyl chain of the inhibitor.

In a further embodiment, there are provided methods for characterizing the physical aspects of the enzyme which allow heterologous agents to enter, interact with, or otherwise perturb the native structure of the enzyme. The invention describes the unpredicted presence of a second, alternate route of entry to the active site distinct from the cellular membrane. Further, the invention provides for the identification of a src homology (SH3) binding domain on an aqueous surface of the enzyme at a quaternary interface.

In a still further embodiment, there are provided methods for identifying, characterizing, and optimizing agents that interact with the internal channels of FAAH and therefore stimulate, inhibit, relocalize, stabilize, or destabilize FAAH and/or its activity. Such an agent might interact with FAAH, for example, at the active site, the substrate binding pocket, the membrane port, the cytosolic port, the dimerization tunnel, the membrane-binding domain, the alkyl tunnel, the head group tunnel, and the like. In another embodiment, there are provided methods for identifying agents that interact with the SH3-binding domain and the surface helix-loop-helix. Such an agent may be further identified, characterized, or optimized by comparison with the MAFP molecule. An interaction between FAAH and such an agent may be investigated with the aid of manual or computerized simulation of the interaction between the agent and FAAH. Such a simulation may then be optimized based on the structure of FAAH and observed complementarities and incompatibilities between FAAH and the agent under consideration. The effects of such an agent may then be determined by obtaining the agent and bringing it in contact with FAAH to measure its effects.

In yet another embodiment, there are provided methods for the elucidation of the three-dimensional structure of a protein or protein complex which is structurally related to FAAH. In this embodiment, there is provided a method by which a molecule or complex may be crystallized and X-ray diffraction data obtained from the crystal. The resulting diffraction data may then be compared with the present known three-dimensional structure of FAAH, and the structure of the molecule may be determined by the method of molecular replacement.

In a further embodiment, there are provided methods for identifying the domain of FAAH responsible for the enzyme's association with membranes (membrane-binding domain, MBD). In this embodiment, there are provided methods wherein the MBD may be combined with other molecules or molecular complexes to cause a novel membrane association in the assembly. Additionally, this method provides a means for removing or mutating the MBD in order to produce FAAH variants that have altered or abolished membrane-binding characteristics.

In yet another embodiment, there are provided methods for altering the active site of FAAH and its substrate recognition mechanism so as to affect a change in the enzyme's substrate selectivity. The method provides for the development of FAAH variants that degrade heterologous compounds. Such FAAH variants may prove useful as novel chemical catalysts for enantioselective amide hydrolysis.

In another embodiment, there are provided methods for screening an agent for the ability to modulate the activity of FAAH, comprising contacting FAAH with the agent to form a FAAH-agent complex and measuring the activity level of the FAAH-agent complex relative to un-complexed FAAH, thereby screening the agent for the ability to modulate the activity of FAAH.

### BRIEF DESCRIPTION OF THE FIGURES

FIG. 1 illustrates an overview of the three-dimensional structure of fatty acid amide hydrolase represented in ribbon diagram format A number of relevant structural features are highlighted. Parts A and B are related by a 90 degree rotation about the vertical axis.

FIG. 2 depicts the active site of FAAH in complex with the arachidonyl inhibitor MAP.

FIG. 3 depicts the predicted membrane-binding cap of FAAH.

FIG. 4 sets forth proposed modular adaptations that convert a soluble enzyme to an integral membrane enzyme based on differences in the structures of FAAH and MAE2.

FIG. 5 depicts exemplary internal channels of FAAH which may be targeted by a subset of agents which interact with the enzyme.

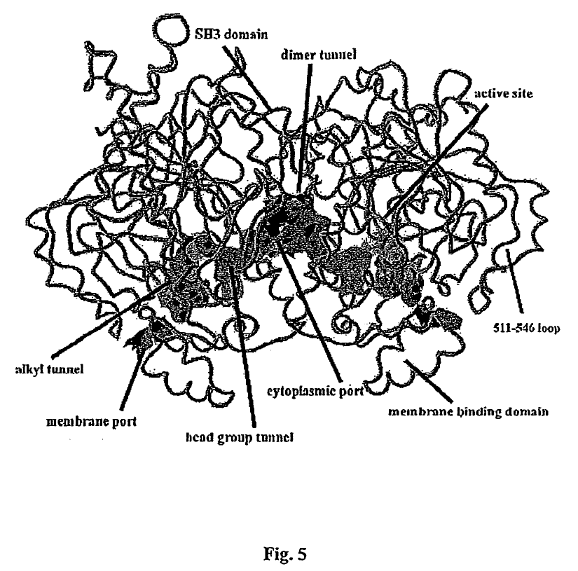

The amino acid sequence of FAAH (SEQ M NO: 1) is included herein.

### DETAILED DESCRIPTION OF THE INVENTION

The present invention provides crystalline mammalian fatty acid amide hydrolase (FAAH) of sufficient quality to produce interpretable X-ray diffraction data. As used herein, the phrase “fatty acid amide hydrolase (FAAH)” includes any protein from any mammalian source that displays a catalytic activity to hydrolyze the amide bond of fatty acid amides and which further contains the amidase signature (AS) sequence. Such a protein may be derived from primary, recombinant, or synthetic sources. As used herein, the term “protein” refers to a protein, polypeptide or peptide.

FAAH has an amino acid sequence as set forth in SEQ ID NO: 1, including conservative variations thereof. As used herein, the phrase “conservative variations” refers to a replacement of an amino acid residue by another, biologically similar amino acid residue. Examples of conservative variations include the substitution of a hydrophobic residue such as isoleucine, valine, leucine or methionine for another, or the substitution of a polar residue for another, such as the substitution of arginine for lysine, glutamic for aspartic acid, or glutamine for asparagine, and the like.

Crystalline FAAH may be produced by combining FAAH protein with a mother liquor (for example, 100 mM sodium citrate pH 5.0, 100 mM lithium sulfate, 8% polyethylene glycol (average molecular weight 6000)) and allowing the mixture to vapor equilibrate with a reservoir of mother liquor for a sufficient time to afford crystals of FAAH. Suitable crystals are introduced into a collimated X-ray source such that the crystal may be rotated and a complete diffraction pattern may be recorded. Such a diffraction pattern may then be analyzed and compared to independent patterns generated from isomorphous crystals derivatized with heavy metals. Heavy metals contemplated for use in the practice of the present invention include, osmium, platinum, and the like.

Alternately, such derivatized crystals may be exposed to X-rays of suitable wavelength(s) such that anomalous signals may be obtained for comparison. Such comparisons may then yield a molecular model of the three-dimensional structure of the FAAH protein. Such a model may be represented by several means, including computer records such as protein data bank (pdb) files (see, e.g., Table II). The invention described herein includes any representation, including but not limited to, binary records, text records, graphic representations, and virtual representations, each either physical or electronic. The present invention also provides for the introduction of an inhibitory substrate analogue to the protein so that its interactions with the protein may be determined.

FAAH crystals may be expected to take a wide variety of forms, all of which are included in the present invention. In one embodiment of the FAAH crystal, the crystal displays primitive monoclinic symmetry P21 and contains sixteen FAAH molecules per asymmetric unit. The monoclinic embodiment further exhibits pseudomerohedral twinning as the crystallographic unit cell axes a and c may be interchanged. In an alternate embodiment, the crystal displays C centered orthorhombic symmetry C2221 and contains eight molecules per asymmetric unit. In a third embodiment, the crystal displays primitive hexagonal symmetry P6322 and contains two molecules per asymmetric unit.

The structure further reveals that the FAAH protein does not exist in the crystal as a monomeric, independent unit, but instead it forms a dimer with simple two-fold symmetry about an axis parallel to the long axis of the monomer. This arrangement results in the concerted presentation of helices 15 through 19 from each monomer on the same face of the assembled dimer. Furthermore, these helices form a protruding, highly hydrophobic plateau on this face of the dimer. As such, this structural motif provides a means for interacting with cellular membranes in a specific and stable manner. This face of the enzyme also displays a route of entry to the active site through which substrates or other agents might pass from the membrane. As used herein, “active site” refers to a region of a FAAH that, as a result of its shape and charge potential, interacts with an agent (including, without limitation, a protein, polypeptide, peptide, nucleic acid, including DNA or RNA, molecule, compound, antibiotic or drug).

A second route of access to the active site is found on a lateral face of the enzyme near the dimer interface. This second port is marked by histidine 449 and possibly tryptophan 445, each from the polypeptide chain of the dimer mate. A possible mechanism exists by which the enzyme may undergo structural rearrangements so that these ports may be opened and closed in a regulated manner in concert with the progress of the enzymatic reaction or as a means for controlling the enzyme's activity. Such a means of control may result from any number of mechanisms, including, but not limited to, the binding of protein partners or ligands. By means of example but not as a complete explanation, such an effect might be triggered by a representative of the SH3-domain class of proteins. The present structure demonstrates that FAAH displays at its apical surface a consensus polyproline sequence for binding SH3-domain proteins, as well as those of the Homer family. This motif resides on the loop between helices eleven and twelve and includes the amino acid sequence proline-proline-leucine-proline. A potential mechanism exists whereby a regulatory protein such as Homer or an exogenously supplied agent may bind the polyproline sequence on FAAH to introduce a conformational change which might alter the cytoplasmic ports of the enzyme or other structural features to change active site geometry or substrate/product transit.

In another embodiment, there are provided methods for identifying agents which interact with the internal channels of FAAH, based on the knowledge of the three-dimensional structure of FAAH, and thereby stimulate, inhibit, relocalize, stabilize, or destabilize FAAH and/or its activity. As used herein, the term “internal channels” refers to several sites within the protein, such as, the active site, the substrate binding pocket, the membrane port, the cytosolic port, the dimerization tunnel, the membrane-binding domain, the alkyl tunnel, the head group tunnel, and the like. The amino acids which comprise the internal channels of FAAH are set forth in Table 5. In still another embodiment, there are provided methods for identifying agents which interact with the SH3-binding domain and the surface helix-loop-helix of FAAH.

Such agents may be identified, developed, and characterized based on an investigation of possible interactions between the agent and the FAAH molecule. This process might occur by visual inspection of the FAAH structure or, more quantitatively, with the aid of various computer algorithms designed for such purposes, such as AUTODOCK, InsightII, and QUANTA. The present invention allows for the virtual binding of candidate agents in silico so that they may be characterized as possible leads for further study. The effects of such agents may then be tested for activity against FAAH by bringing the agent into contact with FAAH and determining any effect on the enzyme's normal function. Such agents identified as candidates by invention methods will aid in identifying lead compounds for further chemical optimization and/or for evaluation in vivo as potential therapeutics. The invention provides that such optimized agents may be used in a pharmacological preparation designed to exert physiological effects when administered to living organisms. Such medically relevant uses for agents identified or optimized with the use of the present invention include, for example, the treatment of pain, sleep, addiction, fertility, anxiety, anorexia, fever, cognitive processes, and the like.

A candidate agent can be any type of molecule, including, for example, a peptide, a peptidomimetic, a polynucleotide, or a small organic molecule, that one wishes to examine for the ability to act as a therapeutic agent, which is an agent that provides a therapeutic advantage to a subject receiving it. It will be recognized that a method of the invention is readily adaptable to a high throughput format and, therefore, the method is convenient for screening a plurality of test agents either serially or in parallel. The plurality of test agents can be, for example, a library of test agents produced by a combinatorial method library of test agents. Methods for preparing a combinatorial library of molecules that can be tested for therapeutic activity are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markland et al., Gene 109:1319, 1991; each of which is incorporated herein by reference); a peptide library (U.S. Pat. No. 5,264,563, which is incorporated herein by reference); a peptidomimetic library (Blondelle et al., Trends Anal. Chem. 14:8392, 1995; a nucleic acid library (O'Connell et al., supra, 1996; Tuerk and Gold, supra, 1990; Gold et al., supra, 1995; each of which is incorporated herein by reference); an oligosaccharide library (York et al., Carb. Res. 285:99128, 1996; Liang et al., Science, 274:1520-1522, 1996; Ding et al., Adv. Expt. Med. Biol., 376:261-269, 1995; each of which is incorporated herein by reference); a lipoprotein library (de Kruif et al., FEBS Lett. 399:232-236, 1996, which is incorporated herein by reference); a glycoprotein or glycolipid library (Karaoglu et al., J. Cell Biol. 130:567-577, 1995, which is incorporated herein by reference); or a chemical library containing, for example, drugs or other pharmaceutical agents (Gordon et al., J. Med. Chem., 37:1385-1401, 1994; Ecker and Crooke, Bio/Technology, 13:351-360, 1995; each of which is incorporated herein by reference). Accordingly, the present invention also provides a therapeutic agent identified by such a method.

The route of administration of a candidate agent will depend, in part, on the chemical structure of the candidate agent. Peptides and polynucleotides, for example, are not particularly useful when administered orally because they can be degraded in the digestive tract. However, methods for chemically modifying peptides, for example, to render them less susceptible to degradation by endogenous proteases or more absorbable through the alimentary tract are well known (see, for example, Blondelle et al., Trends Anal. Chem. 14:83-92, 1995; Ecker and Crooke, Bio/Technology, 13:351-360, 1995; each of which is incorporated herein by reference). In addition, a peptide agent can be prepared using D-amino acids, or can contain one or more domains based on peptidomimetics, which are organic molecules that mimic the structure of peptide domain; or based on a peptoid such as a vinylogous peptoid.

In another embodiment, there are provided pharmaceutical compositions including agents identified by invention methods. Invention pharmaceutical compositions can administered in a variety of ways including, for example, orally or parenterally, such as intravenously, intramuscularly, subcutaneously, intraorbitally, intracapsularly, intraperitoneally, intrarectally, intracisternally or by passive or facilitated absorption through the skin using, for example, a skin patch or transdermal iontophoresis, respectively. Furthermore, the invention pharmaceutical compositions can be administered by injection, incubation, orally or topically, the latter of which can be passive, for example, by direct application of an ointment, or active, for example, using a nasal spray or inhalant, in which case one component of the composition is an appropriate propellant.

The total amount of pharmaceutical composition to be administered in practicing a method of the invention can be administered to a subject as a single dose, either as a bolus or by infusion over a relatively short period of time, or can be administered using a fractionated treatment protocol, in which multiple doses are administered over a prolonged period of time. The pharmaceutical composition can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

In another embodiment, there are provided methods for treating a pathological condition, including administering to a subject in need thereof an invention pharmaceutical composition. In one embodiment, the subject is a mammal. In another embodiment, the subject is human. Pathological conditions that can be effectively treated by invention methods include, for example, anxiety, pain, hunger, sleep, fertility, cognition, immunological disorders, fever, tremor, glaucoma, intestinal disorders, and the like.

In a still further embodiment, there are provided methods for determining the molecular structure of a molecule or molecular complex whose structure is unknown, comprising:

(a) obtaining crystals of the molecule or molecular complex whose structure is unknown;

(b) generating X-ray diffraction data from the crystallized molecule or molecular complex;

(c) comparing the X-ray diffraction data from the molecule or molecular complex with the three dimensional structure determined from the crystalline form of FAAH; and

(d) using molecular replacement analysis to conform the three dimensional structure determined from the crystalline form of FAAH to the X-ray diffraction data from the crystallized molecule or molecular complex. This method allows for the determination, for example, of a protein which is structurally related to FAAH. As used herein, “structurally related” refers to a protein whose structure can be determined by comparing its X-ray diffraction data to the X-ray diffraction data of FAAH. Such a use of the present invention would include the crystallization of the target protein or protein complex and collection of its X-ray diffraction data. The target data may then be interpreted by means of molecular replacement using the FAAH structure as a template. Such methods obviate the need to collect data from derivatized crystals and greatly simplify the process of solving the structure of the target protein.

In a still further embodiment, there are provided methods for engineering proteins with altered physicochemical properties using the structural information obtained from the FAAH crystal. The invention allows for the identification of the membrane-binding domain of the FAAH protein. As such, this domain may be mutated or deleted, in whole or in part, to result in a protein with altered or absent membrane tropism. Further, the identified membrane-binding domain may, in full or in part, be transferred to a heterologous protein so as to affect its membrane tropism. Moreover, the identification of the active site and substrate binding pocket of FAAH allows for the targeted generation of variants with altered substrate specificity and/or altered enzymological properties. Such variants may prove useful as catalysts of amide hydrolysis unrelated to the fatty acid amides, and as such may have chemical or industrial applications.

The invention will now be described in greater detail by reference to the following non-limiting example.

### EXAMPLE

### Crystallization

Protein crystals were obtained by recombinantly expressing rat FAAH with 29 amino acids at the amino-terminus deleted. Though the deleted region is predicted to participate in membrane binding (8), the truncated FAAH variant (residues 30-579) retains the wild-type protein's association with membranes, detergent requirement for solubilization, and ability to degrade fatty acid amides in mammalian cells (15). Crystals of FAAH complexed with methoxy arachidonyl fluorophosphonate, an active site-directed irreversible inhibitor, were obtained in three space groups, P2₁ with sixteen molecules in the assymetric unit, C222₁ with eight molecules in the asymmetric unit, and P6₃22 with two molecules in the asymmetric unit (Materials and Methods (16)). The structure of the FAAH-inhibitor complex was first determined to 3.3 Å using a combination MIRAS/MAD phasing strategy followed by phase extension with multi-crystal and NCS averaging (Table 1). The resulting electron density maps displayed density for over 90% of the side chains with no main chain breaks, yielding an exceptional search model for molecular replacement phasing of twinned monoclinic data. The final model, built in P2₁ at 2.8 Å resolution, was of excellent quality.

### Data Collection

Table 1 includes a summary of data collection and refinement statistics. Initial phases were calculated with DmMulti (24), incorporating all MAD and MIRAS contributions. Ramachandran distributions of amino acids in the final model were calculated with PROCHECK (24) from a single protein chain after phase refinement under tight NCS restraints. Side chains for which no density was observed were set to zero occupancy during refinement, and no sigma-based rejection of reflections was employed. Numbers in parentheses refer to data in the highest resolution shell. Two residues were in disallowed regions: glutamate 122, which is in the i+1 position of a gamma turn, and glutamine 189, which is also in a tight turn immediately followed by a second turn. Table 2 includes the complete data collection and refinement statistics.

TABLE 1

| | TWIN | MAD | | | |
|---|---|---|---|---|---|
| Data set | T1 | Ta peak | Ta inflection | Os peak | Remote |
| Beam line | ALS 5.0.2 | SSRL 9-2 | SSRL 9-2 | SSRL 9-2 | SSRL 9-2 |
| Wavelength (Å) | 0.97910 | 1.255 | 1.2554 | 1.14025 | 0.925 |
| Space group | P2₁ | C222₁ | C222₁ | C222₁ | C222₁ |
| Resolution (Å) | 50-2.8 | 65-3.3 | 65-3.3 | 60-3.3 | 48-3.3 |
| Unique reflections | 173615 | 81567 | 81574 | 81609 | 81511 |
| Redundancy | 2.1 | 14.5 | 14.5 | 14.5 | 14.5 |
| Completeness* | 76 (79) | 99.7 (99.1) | 99.7 (99.2) | 99.8 (99.5) | 99.7 (98.7) |
| I/(σI)* | 4.4 (2.7) | 8.2 (2.3) | 6.9 (1.2) | 7.6 (1.7) | 8.5 (2.2) |
| Rsym (%)* | 10.4 (26.4) | 7.9 (33.3) | 9.7 (63.2) | 8.5 (45.5) | 7.4 (33.4) |

Phasing from Ta₆Cl₁₂•OsCl₃ MAD data

| | |
|---|---|
| Resolution (Å) | 96-3.3 |
| Number of Os sites | 8 |
| Number of Ta sites | 0 |
| Os peak isomorphous phasing power (centric/acentric) | 0.948/1.43 |
| Os peak anomalous phasing power (acentric) | 2.18 |
| Remote isomorphous phasing power (centric/acentric) | 0.288/0.471 |
| Remote anomalous phasing power (acentric) | 1.88 |
| Ta inflection anomalous phasing power (acentric) | 0.871 |
| Ta peak (reference) anomalous phasing power (acentric) | 1.02 |
| Combined figure of merit before NCS and multi-crystal | 0.47/0.60 |
| averaging (3.3 Å) (centric/acentric) | |
| Twinning operator | (l, −h, k) |
| Twinning factor (α) | 0.28 |

Refinement against detwinned data set T1

| | |
|---|---|
| Resolution (Å) | 50-2.8 |
| Reflections used | 173615 |
| Test reflections | 9297 |
| Number of nonhydrogen atoms | 65744 |
| R_{work} (%) | 19.7 |
| R_{free} (%) | 24.9 |
| Average B value (Å²) | 44.829 |
| MAFP average B value (Å²) | 64.26 |
| rmsd bond length (Å) | 0.079 |
| rmsd bond angle (°) | 2.500 |

Ramachandran distributions (%)

| | |
|---|---|
| Most favored | 79.9 |
| Additionally allowed | 17.7 |
| Generously allowed | 2.0 |
| Disallowed^{†} | 0.4 |

TABLE 2

| | NATIVES | | | | TWIN | SAD | | |
|---|---|---|---|---|---|---|---|---|
| Data set | N1 | N2 | N3 | H1 | T1 | (NH₃)₂PtCl₂ | OsCl₃ | Ta peak |
| Beam line | SSRL 9-2 | SSRL 11-1 | SSRL 11-1 | SSRL 9-2 | ALS 5.0.2 | SSRL 11-1 | SSRL 9-2 | SSRL 9-2 |
| Wavelength (Å) | 1.03317 | 1.07185 | 1.07185 | 0.97946 | 0.97910 | 1.07185 | 1.13973 | 1.255 |
| Space group | C222₁ | C222₁ | C222₁ | P6₃22 | P2₁ | C222₁ | C222₁ | C222₁ |
| Resolution (Å) | 60-5.0 | 70-4.0 | 64.5-3.8 | 50-3.5 | 50-2.8 | 70-4.0 | 63-4.0 | 65-3.3 |
| Unique | 19722 | 46254 | 53415 | 18446 | 173615 | 45171 | 46191 | 81567 |
| reflections | | | | | | | | |
| Redundancy | 4.1 | 4.8 | 4.7 | 20 | 2.1 | 9.5 | 9.5 | 14.5 |
| Completeness* | 83 (83) | 99.7 (100) | 99.7 (99.7) | 99.9 (99.9) | 76 (79) | 97.7 (98.6) | 99.3 (99.0) | 99.7 (99.1) |
| I/(σI)* | 6.0 (2.8) | 5.9 (2.6) | 9.1 (4.0) | 4.6 (1.9) | 4.4 (2.7) | 4.8 (1.9) | 7.0 (3.2) | 8.2 (2.3) |
| Rsym (%)* | 10.5 (27.2) | 9.7 (26.5) | 6.7 (18.6) | 12.6 (48.8) | 10.4 (26.4) | 9.6 (39.4) | 9.4 (22.4) | 7.9 (33.3) |

| Phasing from MIRAS data | N1 | N2 | N3 |
|---|---|---|---|
| Resolution (Å) | 48-5.0 | 48-4.0 | 48-4.0 |
| Number of Pt sites | 13 | 13 | 13 |
| Number of Os sites | 8 | 8 | 8 |
| Pt isomorphous phasing power (centric/acentric) | 0.062/0.173 | 0.107/0.178 | 0.123/0.181 |
| Pt anomalous phasing power (acentric) | 0.583 | 0.987 | 0.548 |
| Os isomorphous phasing power (centric/acentric) | 0.555/0.692 | 0.436/0.638 | 0.408/0.558 |
| Os anomalous phasing power (acentric) | 0.767 | 0.736 | 0.739 |

Phasing from Ta₆Cl₁₂•OsCl₃ MAD data

| | |
|---|---|
| Resolution (Å) | 96-3.3 |
| Number of Os sites | 8 |
| Number of Ta sites | 0 |
| Os peak isomorphous phasing power (centric/acentric) | 0.948/1.43 |
| Os peak anomalous phasing power (acentric) | 2.18 |
| Remote isomorphous phasing power (centric/acentric) | 0.288/0.471 |
| Remote anomalous phasing power (acentric) | 1.88 |
| Ta inflection anomalous phasing power (acentric) | 0.871 |
| Ta peak (reference) anomalous phasing power (acentric) | 1.02 |
| Combined figure of merit before NCS and multi-crystal averaging (3.3 Å) | 0.47/0.60 |
| (centric/acentric) | |

Refinement against detwinned data set T1

Resolution (Å)
Reflections used
Test reflections
Number of nonhydrogen atoms
R_{work} (%)
R_{free} (%)
Average B value (Å²)
MAFP average B value (Å²)
rmsd bond length (Å)
rmsd bond angle (°)
Ramachandran distributions (%)
Most favored
Additionally allowed
Generously allowed
Disallowed^{†}
Twinning operator
Twinning factor (α)
*Numbers in parentheses refer to data in the highest resolution shell.
^{†}Two residues were in disallowed regions: glutamate 122, which is in the i + 1 position of a gamma turn, and glutamine 189, which is also in a tight turn immediately followed by a second turn.

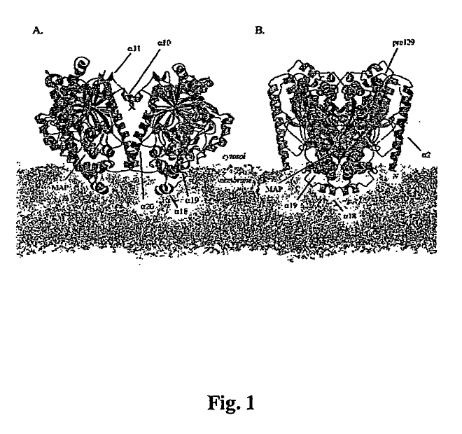

The crystal structure of FAAH reveals a dimeric enzyme (FIG. 1), consistent with chemical crosslinking studies indicating that the enzyme is a dimer in solution (15). The dimer interface buries approximately 1560 Å² of molecular surface area per monomer (17). The protein core is characterized by a twisted beta sheet consisting of eleven mixed strands which is in turn surrounded by twenty-four alpha helices of varying lengths. The overall fold of the monomer closely resembles that of malonamidase (MAE2) from the nitrogen fixing bacterium Bradyrhizobium japonicum (12) and peptide amidase (PAM) from Stenotrophomonas maltophilia (REF: Labahn J, Neumann S, Buldt G, Kula M, Granzin J. J Mol Biol 2002 Oct. 4; 322(5): 1053).

However, key structural features make FAAH unique, and these will be discussed below. Further, these variations can be quantified by measuring the root mean squared deviation (rmsd) between FAAH and its bacterial homologues (Tables 3 and 4). If a comparison is drawn between domains of the proteins which obey amino acid sequence alignment, the rmsd difference increases as more atoms are included in the calculation. This is true both in the case of restricting the calculation to an expanding sphere centered on the active site and in the case of expanding the calculation from Cαs to all atoms. In any case, it is also of note that only a fraction of FAAH can be included in the calculation due to sequence considerations, 42% in the case of PAM and 36% in the case of MAE2 (Table 3). Further, if protein fold topology is taken into consideration, the comparable fractions of FAAH which can be included fall even further, to 22% for PAM and 34% for MAE2 (Table 4).

TABLE 3
Average rmsd_{xyz} of sequence-based equivalent residue
ranges after superposition of catalytic triad Cαs. The number
of amino acid residues included in the calculation is
indicated.

| | 5 Å | 10 Å | 20 Å | 100 Å |
|---|---|---|---|---|
| PAM-CHY* | | | | |
| Cα (222) | 1.217 | 4.443 | 6.442 | 8.525 |
| BACKBONE | 2.361 | 4.044 | 6.452 | 8.494 |
| ALL ATOMS | 2.195 | 4.486 | 6.752 | 8.737 |
| MAE2* | | | | |
| Cα (191) | 0.433 | 2.191 | 5.487 | 9.092 |
| BACKBONE | 1.272 | 2.088 | 4.634 | 8.702 |
| ALL ATOMS | 1.253 | 2.363 | 4.827 | 8.783 |

TABLE 4
Average rmsd_{xyz} of topology-based equivalent
residue ranges. The number of amino acid residues
included in the calculation is indicated.

| | 5 Å | 10 Å | 20 Å | 100 Å |
|---|---|---|---|---|
| PAM-CHY* | | | | |
| Cα (116) | 0.415 | 1.136 | 5.473 | 5.449 |
| BACKBONE | 0.553 | 1.126 | 5.350 | 5.488 |
| ALL ATOMS | 0.504 | 1.126 | 5.147 | 5.290 |
| MAE2* | | | | |
| Cα (178) | 0.561 | 1.044 | 1.146 | 1.384 |
| BACKBONE | 0.661 | 1.028 | 1.221 | 1.429 |
| ALL ATOMS | 0.606 | 1.134 | 1.341 | 1.664 |

*Insertions/deletions and non-identical amino acid side chains are not included in the calculation. PAM-CHY is the complex of peptide amidase and the inhibitor chymostatin.

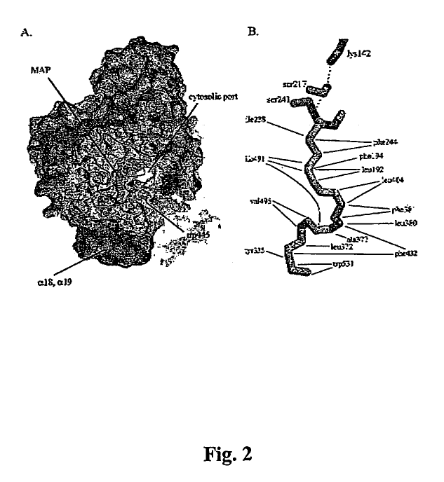

The active site of FAAH was identified based on the location of core catalytic residues defined previously by mutagenesis (10, 18) and from the density of the inhibitor adduct methoxy arachidonyl phosphonate (MAP) (FIG. 2). The catalytic nucleophile, serine 241, is covalently bonded to the phosphorous of the MAP molecule, and the neighboring density can be modeled to accommodate the arachidonyl chain in an energetically favorable conformation (FIG. 2A). As observed for MAE2, the serine nucleophile in FAAH forms part of an unusual serine-serine-lysine catalytic triad with serine 217 and lysine 142 (FIG. 2B), and the relative orientation of the catalytic residues is nearly superimposable between the structures of FAAH and MAE2 (rmsd=0.27 Å). In contrast, the substrate-binding pockets of FAAH and MAE2 differ significantly. The tunnel leading from the surface of FAAH and containing the buried arachidonyl chain is lined with a preponderance of aromatic and aliphatic amino acids (FIG. 2B), many of which arise from sequence insertions in FAAH relative to MAE2. These residues include isoleucine 491, identified by UV cross-linking and mutagenesis studies to participate in substrate recognition (14). In contrast, the active site of MAE2 is lined with mostly hydrophilic amino acids that accommodate its soluble substrate, malonamide (12).

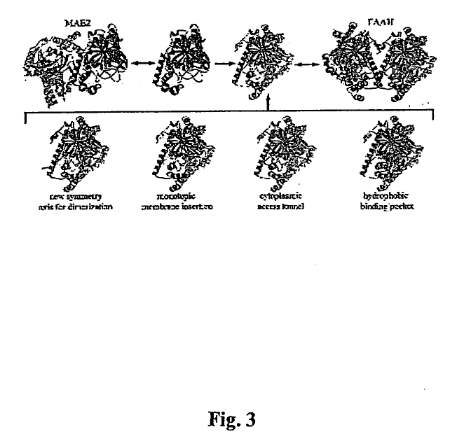

Amino acids 410 to 438, another sequence insertion in FAAH relative to MAE2, form a helix-turn-helix motif that interrupts the AS fold. The two helices, α18 and α19, cap the active site and present several hydrophobic residues that likely compose FAAH's membrane binding face (FIGS. 1, 3A). The N-terminus of the intact enzyme, predicted by sequence analysis to form an additional membrane-binding helix (amino acids 7-29), would be properly positioned to reinforce the membrane interactions of the α18 and α19 membrane cap (FIG. 1B).

A potential substrate entryway is adjacent to α18 and α19, and the arachidonyl chain of MAP contacts phenylalanine 432 of α18, which may indicate direct access between the FAAH active site and the hydrophobic membrane bilayer. The putative substrate entry is amphipathic in nature with hydrophobic residues covering three sides of the rim and charged residues arginine 486 and aspartate 403 completing the remaining side (FIG. 2A, 3B). This arrangement of residues may accommodate the admission and movement of polar fatty acid amide head groups towards the active site. Overall, the intimate relationship between the membrane binding surface and active site of FAAH is similar to that of squalene cyclase (19) and prostaglandin H₂ synthase (20) which also act upon lipid soluble substrates and have hydrophobic caps surrounding their respective active site entrances. In all three enzymes, the hydrophobic cap is surrounded by basic amino acids (FIG. 3B) that may interact with negatively charged phospholipids.

A second major tunnel emerges from the active site at an approximately 80 degree angle from the arachidonyl-filled cavity. This tunnel bifurcates to create both a solvent-exposed cytosolic port and a route blocked by tryptophan 445 (FIG. 2A), a residue that forms a lock-and-key intersubunit contact. Thus, the FAAH active site appears to simultaneously access both the aqueous environment of the cytoplasm and the lipid milieu of the bilayer. This architecture may provide an exit route to the cytosol for the polar amine substituents liberated from the fatty acid amide substrates and could also provide entry for a water molecule required for deacylation of the FAAH-fatty acyl intermediate (18).

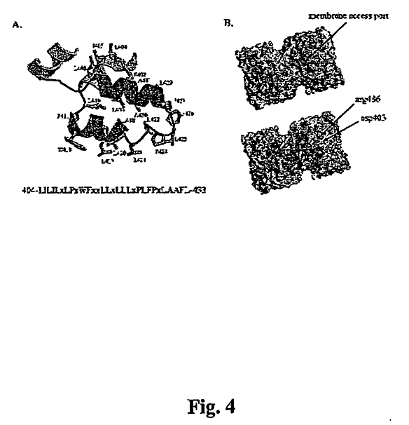

Overall, a comparison of FAAH's structure to that of the soluble AS enzyme MAE2 reveals how a single member of a large clade of proteins has adapted to perform a specialized cellular function through the addition of discrete folding modules and without gross changes in catalytic mechanism or fold architecture (FIG. 4). For example, although FAAH and MAE2 dimerize about roughly the same face, they have different relative monomer orientations. These distinct quanternary orientations produce an antiparallel and parallel alignment of the MAE2 and FAAH monomer active site entrances, respectively. The parallel orientation of FAAH monomers has important biological implications, as it should permit both subunits to function concurrently by recruiting substrates from the same membrane. Furthermore, this parallel alignment places another key structural module, the α18-α19 hydrophobic cap, on the same face of the dimer, thereby enhancing membrane binding (FIG. 4). Additional sequence insertions in the FAAH protein account for other specialized features of the enzyme's structure, including its cytoplasmic channel and apolar substrate binding pocket (FIG. 4).

The intimate relationship between the active site of FAAH and cell membranes revealed by the enzyme's structure raises the possibility that fatty acid amides need not be transported through aqueous cellular compartments in order to proceed from site of action to site of degradation. As a consequence, proper endocannabinoid tone may rely on both the expression levels of FAAH and its localization relative to CB receptor systems in vivo (21). Finally, the structure of FAAH may facilitate the design of inhibitors that could have therapeutic benefit for a variety of nervous system disorders (22), including not only agents that bind to the enzyme acyl chain pocket, but also compounds that target the active site via the cytoplasmic channel. This latter class of inhibitors might display exceptional selectivity for FAAH relative to the more than one hundred lipid hydrolases present in the human proteome.

FIG. 1 depicts the structure of the integral membrane protein fatty acid amide hydrolase (FAAH). The enzyme is a homodimer assembled from 63 kDa subunits. The inhibitor adduct methoxy arachidonyl phosphonate (MAP) is depicted in the active site with van der Waal surfaces rendered in yellow (carbon), gray (phosphorous), and red (oxygen). (A) Front view of the enzyme, highlighting the central twisted beta sheet that forms the core of the structure. (B) Side view produced by a 90° rotation from view A. The amino (N) and carboxyl (C) termini of the green subunit are labeled. Proline 129 is indicated. A genetic polymorphism conferring substitution of threonine at this position has been implicated in drug abuse and renders the enzyme more vulnerable to proteolytic attack (25). All figures were produced with Molscript (26), GRASP (23), and Raster3D (27). The membrane model was generated by Molecular Dynamics simulation of a palmitoyloleoylphosphatidylethanolamine (POPE) bilayer (28).

FIG. 2 depicts the active site of FAAH in complex with the arachidonyl inhibitor MAP. (A) The FAAH dimer is shown with the protein surface rendered in gray (hydrophilic) and green (hydrophobic) for one subunit and pale yellow for its mate. A portion of the protein surface has been removed to highlight the continuous internal channel leading from the membrane binding face at arginine 486 (blue) and aspartate 403 (red) to the active site and on to the cytosolic port. Electron density corresponding to the arachidonyl inhibitor is shown in violet and lies in the hydrophobic (green) substrate-binding pocket. Tryptophan 445 from the dimer mate is rendered in van der Waals surface and colored cyan to demonstrate the effective plugging of this potential port. (B) Aromatic and aliphatic residues in the substrate binding pocket surrounding the arachidonyl chain and their interactions are indicated. Also shown is the unusual serine-serine-lysine catalytic triad of FAAH (ser241-ser217-ser241), with the serine 241 nucleophile covalently bonded to the phosphorus atom of the MAP inhibitor.

FIG. 3 depicts the predicted membrane-binding cap of FAAH. (A) The hydrophobic helices α18 and α19 that comprise the putative membrane-binding cap are shown in green. The primary sequence of this domain (404-433) is indicated using amino acid one letter code (29) except for hydrophilic amino acids, which are indicated by x. Five of these seven hydrophilic residues are arginine or lysine; the remaining two are serines. (B) The molecular surface of FAAH viewed from the membrane face; the observed structure demonstrates the presence of a hydrophobic cap (top, green), surrounded primarily by positive electrostatic potential (bottom; blue, basic; red, acidic). The entrance to the active site from the membrane face is indicated, as are arginine 486 and aspartate 403, which form one side of this access port.

FIG. 4 sets forth proposed modular adaptations that convert a soluble enzyme to an integral membrane enzyme based on differences in the structures of FAAH and MAE2. The soluble enzyme, if oligomeric, reorganizes so that all active sites can concurrently access the bilayer for maximal efficiency. The addition of discrete structural elements, represented here in red, confer the new oligomerization domain (tryptophan 445 and residues 299-314; lower left panel) as well as a membrane binding face (lower middle left panel) and cytoplasmic access channel (lower middle right panel). The final monotopic integral membrane enzyme must also undergo mutation of key substrate binding residues in the active site to effectively recruit its hydrophobic targets from the lipid bilayer (lower right panel).

TABLE 5
Residues expected to line the interior channels of FAAH. The coordinates of
the Cαs in the present structure are provided as a guide only.

| | | | | | | | | | | | | |
|---|---|---|---|---|---|---|---|---|---|---|---|---|
| ATOM | 0 | CA | LYS | A | 142 | 12.558 | −31.943 | 29.679 | 0.00 | 0.00 | A | C |
| ATOM | 0 | CA | SER | A | 190 | 17.871 | −20.245 | 32.364 | 0.00 | 0.00 | A | C |
| ATOM | 0 | CA | MET | A | 191 | 17.760 | −22.751 | 29.499 | 0.00 | 0.00 | A | C |
| ATOM | 0 | CA | LEU | A | 192 | 19.766 | −20.186 | 27.640 | 0.00 | 0.00 | A | C |
| ATOM | 0 | CA | SER | A | 193 | 22.793 | −22.387 | 27.483 | 0.00 | 0.00 | A | C |
| ATOM | 0 | CA | PHE | A | 194 | 24.110 | −25.405 | 25.603 | 0.00 | 0.00 | A | C |
| ATOM | 0 | CA | SER | A | 217 | 19.643 | −27.223 | 27.046 | 0.00 | 0.00 | A | C |
| ATOM | 0 | CA | THR | A | 236 | 12.999 | −27.716 | 22.998 | 0.00 | 0.00 | A | C |
| ATOM | 0 | CA | ILE | A | 238 | 15.579 | −22.197 | 19.986 | 0.00 | 0.00 | A | C |
| ATOM | 0 | CA | GLY | A | 239 | 19.319 | −22.114 | 19.282 | 0.00 | 0.00 | A | C |
| ATOM | 0 | CA | GLY | A | 240 | 20.185 | −25.826 | 19.177 | 0.00 | 0.00 | A | C |
| ATOM | 0 | CA | SER | A | 241 | 19.006 | −27.246 | 22.499 | 0.00 | 0.00 | A | C |
| ATOM | 0 | CA | PHE | A | 244 | 23.393 | −30.169 | 20.863 | 0.00 | 0.00 | A | C |
| ATOM | 0 | CA | THR | A | 257 | 9.128 | −26.186 | 12.131 | 0.00 | 0.00 | A | C |
| ATOM | 0 | CA | ASN | A | 259 | 4.442 | −24.354 | 14.717 | 0.00 | 0.00 | A | C |
| ATOM | 0 | CA | LEU | A | 261 | 3.547 | −28.979 | 17.435 | 0.00 | 0.00 | A | C |
| ATOM | 0 | CA | SER | A | 262 | 1.827 | −27.120 | 20.226 | 0.00 | 0.00 | A | C |
| ATOM | 0 | CA | LYS | A | 263 | 3.541 | −25.099 | 22.924 | 0.00 | 0.00 | A | C |
| ATOM | 0 | CA | SER | A | 264 | 0.514 | −24.586 | 24.939 | 0.00 | 0.00 | A | C |
| ATOM | 0 | CA | LEU | A | 266 | 5.685 | −24.877 | 27.877 | 0.00 | 0.00 | A | C |
| ATOM | 0 | CA | GLY | A | 268 | 9.936 | −20.496 | 29.403 | 0.00 | 0.00 | A | C |
| ATOM | 0 | CA | CYS | A | 269 | 11.633 | −17.639 | 31.102 | 0.00 | 0.00 | A | C |
| ATOM | 0 | CA | VAL | A | 270 | 10.747 | −14.992 | 28.633 | 0.00 | 0.00 | A | C |
| ATOM | 0 | CA | TYR | A | 271 | 7.552 | −14.969 | 26.504 | 0.00 | 0.00 | A | C |
| ATOM | 0 | CA | GLY | A | 272 | 6.363 | −13.140 | 23.340 | 0.00 | 0.00 | A | C |
| ATOM | 0 | CA | GLN | A | 273 | 9.614 | −13.020 | 21.432 | 0.00 | 0.00 | A | C |
| ATOM | 0 | CA | THR | A | 274 | 8.660 | −14.154 | 17.873 | 0.00 | 0.00 | A | C |
| ATOM | 0 | CA | ALA | A | 275 | 11.611 | −13.454 | 15.736 | 0.00 | 0.00 | A | C |
| ATOM | 0 | CA | VAL | A | 276 | 13.533 | −16.679 | 15.922 | 0.00 | 0.00 | A | C |
| ATOM | 0 | CA | GLN | A | 277 | 11.336 | −19.629 | 16.496 | 0.00 | 0.00 | A | C |
| ATOM | 0 | CA | LEU | A | 278 | 10.970 | −21.950 | 19.360 | 0.00 | 0.00 | A | C |
| ATOM | 0 | CA | ASP | A | 306 | −4.793 | −26.348 | 20.414 | 0.00 | 0.00 | A | C |
| ATOM | 0 | CA | THR | A | 308 | −3.495 | −22.832 | 16.561 | 0.00 | 0.00 | A | C |
| ATOM | 0 | CA | VAL | A | 309 | −1.332 | −25.679 | 15.400 | 0.00 | 0.00 | A | C |
| ATOM | 0 | CA | TYR | A | 335 | 32.027 | −18.555 | 10.993 | 0.00 | 0.00 | A | C |
| ATOM | 0 | CA | LEU | A | 372 | 23.776 | −14.742 | 12.937 | 0.00 | 0.00 | A | C |
| ATOM | 0 | CA | GLU | A | 373 | 24.892 | −11.299 | 14.242 | 0.00 | 0.00 | A | C |
| ATOM | 0 | CA | SER | A | 376 | 19.587 | −14.121 | 15.269 | 0.00 | 0.00 | A | C |
| ATOM | 0 | CA | ALA | A | 377 | 21.364 | −12.481 | 18.109 | 0.00 | 0.00 | A | C |
| ATOM | 0 | CA | LEU | A | 380 | 17.049 | −12.894 | 20.827 | 0.00 | 0.00 | A | C |
| ATOM | 0 | CA | PHE | A | 381 | 18.126 | −9.780 | 22.708 | 0.00 | 0.00 | A | C |
| ATOM | 0 | CA | PHE | A | 388 | 15.472 | −12.482 | 30.704 | 0.00 | 0.00 | A | C |
| ATOM | 0 | CA | LEU | A | 401 | 26.491 | −19.448 | 32.271 | 0.00 | 0.00 | A | C |
| ATOM | 0 | CA | GLY | A | 402 | 29.874 | −17.666 | 32.101 | 0.00 | 0.00 | A | C |
| ATOM | 0 | CA | ASP | A | 403 | 29.566 | −13.889 | 31.936 | 0.00 | 0.00 | A | C |
| ATOM | 0 | CA | LEU | A | 404 | 26.384 | −13.628 | 29.944 | 0.00 | 0.00 | A | C |
| ATOM | 0 | CA | ILE | A | 407 | 27.407 | −8.419 | 30.439 | 0.00 | 0.00 | A | C |
| ATOM | 0 | CA | ARG | A | 428 | 32.279 | −10.875 | 16.349 | 0.00 | 0.00 | A | C |
| ATOM | 0 | CA | LEU | A | 429 | 32.511 | −9.785 | 19.879 | 0.00 | 0.00 | A | C |
| ATOM | 0 | CA | PHE | A | 432 | 27.625 | −9.601 | 20.031 | 0.00 | 0.00 | A | C |
| ATOM | 0 | CA | TRP | A | 445 | 9.219 | −4.401 | 13.406 | 0.00 | 0.00 | A | C |
| ATOM | 0 | CA | GLN | A | 448 | 10.948 | −9.045 | 11.948 | 0.00 | 0.00 | A | C |
| ATOM | 0 | CA | HIS | A | 449 | 10.701 | −7.903 | 8.430 | 0.00 | 0.00 | A | C |
| ATOM | 0 | CA | ILE | A | 451 | 13.771 | −11.849 | 8.960 | 0.00 | 0.00 | A | C |
| ATOM | 0 | CA | GLU | A | 452 | 10.849 | −12.835 | 6.872 | 0.00 | 0.00 | A | C |
| ATOM | 0 | CA | MET | A | 453 | 12.858 | −11.529 | 3.862 | 0.00 | 0.00 | A | C |
| ATOM | 0 | CA | ARG | A | 455 | 13.826 | −16.561 | 4.859 | 0.00 | 0.00 | A | C |
| ATOM | 0 | CA | GLN | A | 456 | 12.307 | −15.855 | 1.397 | 0.00 | 0.00 | A | C |
| ATOM | 0 | CA | PRO | A | 484 | 29.797 | −23.817 | 26.313 | 0.00 | 0.00 | A | C |
| ATOM | 0 | CA | GLY | A | 485 | 29.335 | −20.718 | 28.271 | 0.00 | 0.00 | A | C |
| ATOM | 0 | CA | ARG | A | 486 | 32.477 | −19.218 | 26.838 | 0.00 | 0.00 | A | C |
| ATOM | 0 | CA | THR | A | 488 | 28.751 | −18.591 | 22.179 | 0.00 | 0.00 | A | C |
| ATOM | 0 | CA | GLY | A | 489 | 29.978 | −18.362 | 18.590 | 0.00 | 0.00 | A | C |
| ATOM | 0 | CA | ILE | A | 491 | 25.976 | −22.214 | 18.037 | 0.00 | 0.00 | A | C |
| ATOM | 0 | CA | SER | A | 492 | 26.470 | −20.646 | 14.610 | 0.00 | 0.00 | A | C |
| ATOM | 0 | CA | VAL | A | 495 | 20.892 | −21.229 | 15.354 | 0.00 | 0.00 | A | C |
| ATOM | 0 | CA | CYS | A | 499 | 16.329 | −18.125 | 12.495 | 0.00 | 0.00 | A | C |
| ATOM | 0 | CA | LEU | A | 500 | 15.055 | −19.509 | 9.162 | 0.00 | 0.00 | A | C |
| ATOM | 0 | CA | ASP | A | 501 | 12.509 | −21.783 | 10.900 | 0.00 | 0.00 | A | C |
| ATOM | 0 | CA | ILE | A | 530 | 38.543 | −15.553 | 22.466 | 0.00 | 0.00 | A | C |
| ATOM | 0 | CA | TRP | A | 531 | 35.687 | −17.843 | 21.343 | 0.00 | 0.00 | A | C |
| ATOM | 0 | CA | ILE | A | 534 | 38.136 | −21.645 | 24.267 | 0.00 | 0.00 | A | C |
| ATOM | 0 | CA | TRP | A | 556 | 6.679 | −28.127 | 7.026 | 0.00 | 0.00 | A | C |

```
SEQ. ID. NO 1:
    1 mvlsevwttl sgvsgvclac sllsaavvlr wtgrqkarga atrarqkqra sletmdkavq
  61 rfrlqnpdld sealltlpll qlvqklqsge lspeavffty lgkawevnkg tncvtsyltd
121 cetqlsqapr qgllygvpvs lkecfsykgh dstlglslne gmpsesdcvv vqvlklqgav
181 pfvhtnvpqs mlsfdcsnpl fgqtmnpwks skspggssgg egaligsggs plglgtdigg
241 sirfpsafcg icglkptgnr lsksglkgcv ygqtavqlsl gpmardvesl alclkallce
301 hlftldptvp plpfreevyr ssrplrvgyy etdnytmpsp amrralietk qrleaaghtl
361 ipflpnnipy alevlsaggl fsdggrsflq nfkgdfvdpc lgdlililrl pswfkrllsl
421 llkplfprla aflnsmrprs aeklwklqhe iemyrqsvia qwkamnldvl ltpmlgpald
481 lntpgratga isytvlyncl dfpagvvpvt tvtaeddaqm elykgyfgdi wdiilkkamk
541 nsvglpvavq cvalpwqeel clrfmreveq lmtpqkqps
```

### REFERENCES

1. W. A. Devane et al., Science 258, 1946 (1992).
2. B. F. Cravatt et al., Science 268, 1506 (1995).
3. A. Calignano, G. La Rana, A. Guiffrida, D. Piomelli, Nature 394, 277 (1998).
4. F. Rodriguez de Fonseca et al., Nature 414, 209 (2001).
5. A. H. Lichtman, E. G. Hawkins, G. Griffin, B. F. Cravatt, J. Pharmacol. Exp. Ther. 302, 73 (2002).
6. R. I. Wilson, R. A. Nicoll, Science 296, 678 (2002).
7. N. Fukushima, I. Ishii, J. J. Contos, J. A. Weiner, J. Chun, Annu. Rev. Pharmacol. Toxicol. 41, 507 (2001).
8. B. F. Cravatt et al., Nature 384, 83 (1996).
9. B. F. Cravatt et al., Proc. Natl. Acad Sci. U.S.A. 98, 9371 (2001).
10. M. P. Patricelli, M. A. Lovato, B. F. Cravatt, Biochemistry 38, 9804 (1999).
11. K. Gomi, K. Kitamoto, C. Kumagai, Gene 108, 91 (1991).
12. S. Shin et al., EMBO J. 21, 2509 (2002).
13. A. W. Curnow et al., Proc. Natl. Acad. Sci. U.S.A. 94, 11819 (1997).
14. M. P. Patricelli, B. F. Cravatt, Biochemistry 40, 6107 (2001).
15. M. P. Patricelli, H. A. Lashuel, D. K. Giang, J. W. Kelly, B. F. Cravatt, Biochemistry 37, 15177 (1998).
16. Materials and methods are available as supporting online material on Science Online.
17. Buried van der Waals molecular surface area was calculated in GRASP (23).
18. M. P. Patricelli, B. F. Cravatt, Biochemistry 38, 14125 (1999).
19. K. U. Wendt, K. Poralla, G. E. Schultz, Science 277, 1811 (1997).
20. D. Picot, P. J. Loll, R. M. Garavito, Nature 367, 243 (1994).
21. M. Egertova, D. K Giang, B. F. Cravatt, M. R. Elphick, Proc. R. Soc. Lond. B. Biol. Sci. 265, 2081 (1998).
22. D. L. Boger et al., Proc. Natl. Acad. Sci. U.S.A. 97, 5044 (2000).
23. A. Nicholls, K. Sharp, B. Honig, Proteins Struct. Funct. Genet. 11, 282 (1991).
24. Collaborative Computational Project, Number 4, Acta Crysallogr. D50, 760 (1994).
25. J. C. Sipe, K Chiang, A. L. Gerber, E. Beutler, B. F. Cravatt, Proc. Natl. Acad. Sci. U.S.A. 99, 8394 (2002).
26. P. J. Kraulis, J. Appl. Crystallogr. 24, 946 (1991).
27. E. A. Merritt, D. J. Bacon, Methods Enzymol. 277, 505 (1997).
28. D. P. Tieleman, H. J. C. Berendsen, Biophys. J 74, 2786 (1998), http://indigo1.biop.ox.ac.uk/tieleman/downloads.html pope.pdb.
29. A, alanine; F, phenylalanine; L, leucine; L isoleucine; P, proline; W, tryptophan.

While the invention has been described in detail with reference to certain preferred embodiments thereof, it will be understood that modifications and variations are within the spirit and scope of that which is described and claimed.

What is claimed is:

1. A crystallized mammalian fatty acid amide hydrolase (FAAH).

2. The crystallized FAAH of claim 1, wherein said enzyme has an amino acid sequence of SEQ. ID. NO.1, or conservative substitutions thereof.

3. A three-dimensional model for the structure of FAAH at the secondary, tertiary, and quaternary levels.

4. A method for determining the three-dimensional structure of FAAH from the crystals of claim 1, comprising collecting X-ray diffraction data from said crystal, and analyzing said data by multiple isomorphous replacement (MIR) and single- and multiwavelength anomalous diffraction (SAD/MAD), thereby determining the three-dimensional structure of FAAH.

5. A method for determining the molecular structure of a molecule or molecular complex whose structure is unknown, comprising
(a) obtaining crystals of the molecule or molecular complex whose structure is unknown;
(b) generating X-ray diffraction data from the crystallized molecule or molecular complex;
(c) comparing the X-ray diffraction data from the molecule or molecular complex with the three dimensional structure determined from the crystal of claim 1; and
(d) using molecular replacement analysis to conform the three dimensional structure determined from the crystal of claim 1 to the X-ray diffraction data from the crystallized molecule or molecular complex.

6. A method for identifying an agent that interacts with an internal channel of FAAH, comprising performing manual or computer assisted fitting analysis of the agent with the three-dimensional model of claim 3, thereby identifying an agent that interacts with an internal channel of FAAH.

7. The method of claim 6, wherein the internal channel is the active site.

8. The method of claim 7, wherein the active site comprises amino acid residues 142, 217, 218, 236, 238, 239, 240 and 241.

9. The method of claim 6, wherein the internal channel is the substrate binding pocket.

10. The method of claim 9, wherein the substrate binding pocket comprises amino acid residues 491, 495, 335, 372, 377, 194, and 244.

11. The method of claim 9, wherein the interaction of the agent with the substrate binding pocket is identified, optimized, or compared to the position of arachidonyl inhibitor MAFP.

12. The method of claim 6, wherein the internal channel is the membrane port of FAAH.

13. The method of claim 12, wherein the membrane port comprises amino acid residues 403, 407, 428, 429, 486, 530, and 534.

14. The method of claim 6, wherein the internal channel is the cytosolic port of FAAH.

15. The method of claim 14, wherein the cytosolic port comprises amino acid residues 266, 271, 272, 445, 449, and 453.

16. The method of claim 6, wherein the internal channel is the dimerization tunnel of FAAH.

17. The method of claim 16, wherein the dimerization tunnel comprises amino acid residues 257, 259, 261, 262, 263, 264, 306, 308, 309, 448, 451, 452, 455, 456, 499, 500, 501, and 556.

18. The method of claim 6, wherein the internal channel is the membrane-binding domain of FAAH.

19. The method of claim 18, wherein the membrane-binding domain comprises amino acid residues 383-440.

20. The method of claim 6, wherein the internal channel is the head group tunnel of FAAH.

21. The method of claim 20, wherein the head group tunnel comprises amino acid residues 268, 269, 270, 273, 274, 275, 276, 277, and 278.

22. The method of claim 6, wherein the internal channel is the alkyl tunnel of FAAH.

23. The method of claim 22, wherein the alkyl tunnel comprises amino acid residues 190-194, 244, 335, 372, 373, 376, 377, 380, 381, 388, 401, 402, 404, 432, 484, 485, 488, 489, 491, 492, 495, and 531.

24. A method for identifying an agent that interacts with the SH3-binding domain of FAAH, comprising performing manual or computer assisted fitting analysis of the agent with the three-dimensional model of claim 3, thereby identifying an agent that interacts with the SH3-binding domain of FAAH.

25. The method of claim 24, wherein the SH3-binding domain comprises amino acid residues 297-315.

26. A method for identifying an agent that interacts with the surface helix-loop-helix of FAAH, comprising performing manual or computer assisted fitting analysis of the agent with the three-dimensional model of claim 3, thereby identifying an agent that interacts with the surface helix-loop-helix of FAAH.

27. The method of claim 26, wherein the surface helix-loop-helix comprises amino acid residues 511-546.

28. An agent identified by the methods of any one of claims 6-27.

29. A pharmaceutical composition comprising an agent of claim 28 and a pharmaceutically acceptable carrier therefor.

30. A method for treating a pathological condition, comprising administering to a subject in need thereof a pharmaceutical composition according to claim 29, thereby treating the pathological condition.

31. The method of claim 30, wherein said subject is a mammal.

32. The method of claim 31, wherein said mammal is human.

33. The method of claim 30, wherein said pathological condition is anxiety, pain, hunger, sleep, fertility, cognition, immunological disorders, fever, tremor, glaucoma, or intestinal disorders.

34. A method for screening an agent for the ability to modulate the activity of FAAH, comprising contacting FAAH with the agent to form a FAAH-agent complex, and measuring the activity level of said FAAH-agent complex relative to un-complexed FAAH, thereby screening the agent for the ability to modulate the activity of FAAH

35. A method for evaluating the effects of agents identified by claim 34, comprising administering the agents to living cells or a living organism.

36. A method for engineering FAAH variants with altered substrate specificities or kinetics, comprising identifying the substrate binding site of FAAH, and altering said substrate binding site, thereby engineering FAAH variants with altered substrate specificities or kinetics.

37. A method for altering membrane tropism of a heterologous protein, comprising transplanting the entire membrane binding domain of FAAH to the heterologous protein, thereby altering membrane tropism of a heterologous protein.

38. A method for altering membrane tropism of a heterologous protein, comprising transplanting a portion of the membrane binding domain of FAAH to the heterologous protein, thereby altering membrane tropism of a heterologous protein.

39. A method for engineering FAAH variants with altered membrane-binding properties, comprising identifying the membrane-binding domain of FAAH, and altering said membrane-binding domain, thereby engineering FAAH variants with altered membrane-binding properties.


SEQUENCE LISTING (1 sequence)

SEQ ID NO: 1 - protein, 579 aa, Rat
MVLSEVWTTLSGVSGVCLACSLLSAAVVLRWTGRQKARGAATRARQKQRASLETMDKAVQRFRLQNPDLDSEALLTLPLLQLVQKLQSGELSPEAVFFTYLGKAWEVNKGTNCVTSYLTDCETQLSQAPRQGLLYGVPVSLKECFSYKGHDSTLGLSLNEGMPSESDCVVVQVLKLQGAVPFVHTNVPQSMLSFDCSNPLFGQTMNPWKSSKSPGGSSGGEGALIGSGGSPLGLGTDIGGSIRFPSAFCGICGLKPTGNRLSKSGLKGCVYGQTAVQLSLGPMARDVESLALCLKALLCEHLFTLDPTVPPLPFREEVYRSSRPLRVGYYETDNYTMPSPAMRRALIETKQRLEAAGHTLIPFLPNNIPYALEVLSAGGLFSDGGRSFLQNFKGDFVDPCLGDLILILRLPSWFKRLLSLLLKPLFPRLAAFLNSMRPRSAEKLWKLQHEIEMYRQSVIAQWKAMNLDVLLTPMLGPALDLNTPGRATGAISYTVLYNCLDFPAGVVPVTTVTAEDDAQMELYKGYFGDIWDIILKKAMKNSVGLPVAVQCVALPWQEELCLRFMREVEQLMTPQKQPS